(19) United States
(12) Patent Application Publication
(10) Pub. No.: US 2013/0302210 A1
(43) Pub. Date: Nov. 14, 2013

(54) CORROSION INHIBITOR COMPOSITIONS, METHODS FOR MAKING AND METHODS FOR USING

(71) Applicants: Nimeshkumar Kantilal Patel, Bangalore (IN); Baraka Kawawa, Bensalem, PA (US); Glenn Vernon Kenreck, JR., Spring, TX (US); Subbiah Alagarsamy, Bangalore (IN); Gregory Kaplan, Trevose, PA (US); Reblka Mayanglambam Devil, Bangalore (IN); Muthukumar Nagu, Bangalore (IN)

(72) Inventors: Nimeshkumar Kantilal Patel, Bangalore (IN); Baraka Kawawa, Bensalem, PA (US); Glenn Vernon Kenreck, JR., Spring, TX (US); Subbiah Alagarsamy, Bangalore (IN); Gregory Kaplan, Trevose, PA (US); Reblka Mayanglambam Devil, Bangalore (IN); Muthukumar Nagu, Bangalore (IN)

(21) Appl. No.: 13/469,900

(22) Filed: May 11, 2012

(51) Int. Cl. C23F 11/14; C07C 229/30

(52) U.S. Cl. 422/7; 562/565

(57) ABSTRACT

A method for inhibiting corrosion on metallic surfaces in contact with a liquid hydrocarbon media is provided. A corrosion inhibitor composition is dispersed into a liquid hydrocarbon media to form a protective film on the metallic surfaces. The corrosion inhibitor includes a polyamic acid having structure I:

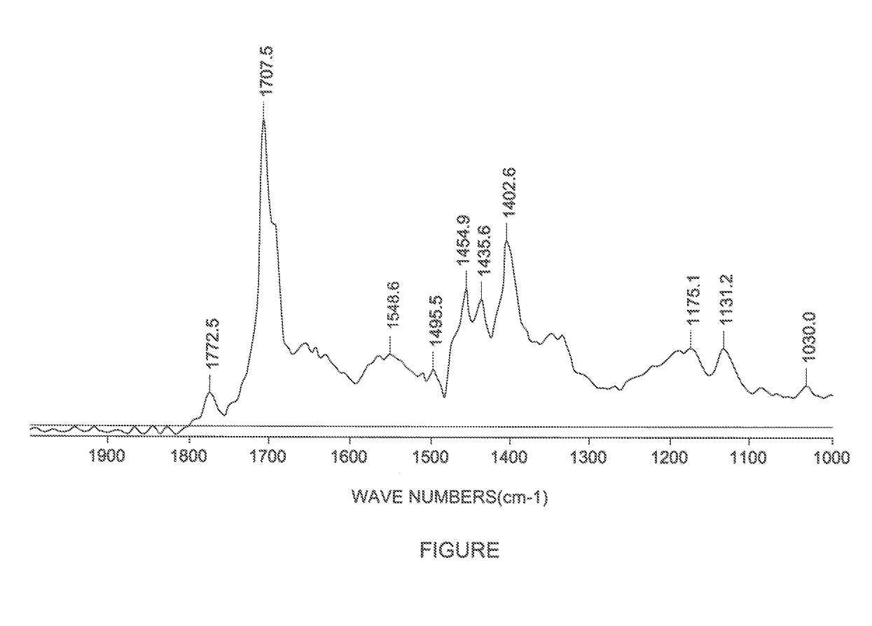

A-HN—(CH₂)₂—[—NH(CH₂)₂—]ₓ—NH-A I

or structure II:

A-HN—(CH₂)₃—[—O—(CH₂)₂—]ₙ—O—(CH₂)₃—NH-A II

wherein A has structure III:

R—CH═CH—CH(CO₂H)—CH₂C(O)— III

and R is a (C₈-C₂₂) alkyl group or a (C₈-C₂₂) alkenyl group, x is an integer from 0 to 6 and n is an integer from 1 to 6. A method for preparing the corrosion inhibitor is also provided.

### FIELD OF THE INVENTION

This invention relates to compositions and methods for inhibiting corrosion and more particularly, to compositions and methods for inhibiting acidic corrosion on metallic surfaces in hydrocarbon processing equipment.

### BACKGROUND OF THE INVENTION

The following description does not admit or imply that the method discussed below is citable as prior art or part of the general knowledge of a person skilled in the art in any particular country.

Corrosion is a problem in many refineries, particularly for overhead systems of crude oil distillation towers, such as piping, vessels, pumps, condensers, heat exchangers, trays and other hydrocarbon processing equipment. Crude oil contains sulfides and chlorides in the form of magnesium chloride and calcium chloride. In refinery processes, crude oil is distilled into light hydrocarbon fractions, which are recovered as overhead fractions from distillation zones. The fractions are cooled, condensed and sent to collection equipment. As the steam in the overhead gas from a tower is condensed into liquid water at the surface of condensation equipment, condensation of hydrogen sulfide and hydrogen chloride formed by hydrolysis of the magnesium chloride and calcium chloride will also occur and become dissolved in the liquid water or water phase. These highly acidic mixtures can corrode the metallic surfaces of the hydrocarbon processing equipment.

Inhibitors have been used to control the corrosiveness of condensed acidic materials in distillation equipment. Generally, nitrogen-based compounds, such as neutralizing amines or amido-amines, imidazolines or pyrimidinium salts and non-nitrogen based compounds, such as dimer-trimer fatty acids have been used as film-forming corrosion inhibitors in overhead equipment. However, these inhibitors do not provide adequate corrosion inhibition over a range of operating conditions, such as temperature ranges or pH ranges, or in the presence of contaminants within the crude oil, such as sulfur and oxygen. In addition, large amounts of the inhibitors are often needed for treatment, which can create negative downstream effects.

U.S. Pat. No. 4,946,626 discloses corrosion inhibitors that are a Diels-Alder adduct of 2,3 and 6,7 poly(allocimene) and an activated olefin, such as maleic anhydride. The adduct may be reacted with a polyamine to form a reaction product with amide groups.

U.S. Pat. No. 5,556,575 discloses polyimide corrosion inhibitors for use in refineries. The imide product is formed from the reaction of hydrocarbyl succinic anhydride and an amine at a temperature of about 180° C.

U.S. Pat. No. 7,897,696 discloses the process for the preparation of polyalkenyl succinic anhydrides, which is formed into a succinimide upon reaction with a polyamine at a temperature of about 150° C. to about 250° C. The succinimide is used as a dispersant in a lubricating oil composition.

What is needed is improved corrosion control for hydrocarbon processing equipment that has a robust performance over a range of operating conditions, has lower treatment cost and uses reduced levels of treatment.

### SUMMARY OF THE INVENTION

In one embodiment, a method for inhibiting corrosion on metallic surfaces in contact with a liquid hydrocarbon media, the method includes dispersing a corrosion inhibitor composition into the liquid hydrocarbon media, wherein the corrosion inhibitor composition includes polyamic acid having structure I:

A-HN—(CH₂)₂—[—NH(CH₂)₂—]ₓ—NH-A I

or structure II:

A-HN—(CH₂)₃—[—O—(CH₂)₂—]ₙ—O—(CH₂)₃—NH-A II

wherein A has structure III:

R—CH═CH—C(CO₂H)—CH₂C(O)— III

and R is a (C₈-C₂₂) alkyl group or a (C₈-C₂₂) alkenyl group, x is an integer from 0 to 6 and n is an integer from 1 to 6.

In another embodiment, a corrosion inhibitor composition including polyamic acid having structure I:

A-HN—(CH₂)₂—[—NH(CH₂)₂—]ₓ—NH-A I

or structure II:

A-HN—(CH₂)₃—[—O—(CH₂)₂—]ₙ—O—(CH₂)₃—NH-A II

wherein A has structure III:

R—CH═CH—C(CO₂H)—CH₂C(O)— III

and R is a (C₈-C₂₂) alkyl group or a (C₈-C₂₂) alkenyl group, x is an integer from 0 to 6 and n is an integer from 1 to 6.

In another embodiment, a method for making a corrosion inhibitor composition including reacting an alkenyl succinic anhydride and a polyamine in a molar ratio of the anhydride to the polyamine of from about 1:1 to about 5:1 at a temperature from about 50° C. to about 95° C., wherein the polyamine has formula IV:

H₂N—(CH₂)₂—[—NH(CH₂)₂—]ₓ—NH₂ IV

or structure V:

H₂N—(CH₂)₃—[—O—(CH₂)₂)ₙ—O—(CH₂)₃—NH₂ V

wherein x is an integer from 0 to 6 and n is an integer from 1 to 6.

The various embodiments provide improved superior and robust corrosion inhibition for metallic surfaces at lower treatment costs without negative downstream effects.

### BRIEF DESCRIPTION OF DRAWINGS

The FIGURE is a ¹³C NMR spectrum for the amic acid prepared in Example 2.

### DETAILED DESCRIPTION OF INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

The terms “comprises”, “comprising”, “includes”, “including”, “has”, “having”, “containing”, “contains”, or any other variation thereof, are intended to cover a non-exclusive inclusion. For example, a process, method, article or apparatus that comprises a list of elements is not necessarily limited to only those elements, but may include other elements not expressly listed or inherent to such process, method, article or apparatus.

In one embodiment, a method for inhibiting corrosion on metallic surfaces in contact with a liquid hydrocarbon media, the method includes dispersing a corrosion inhibitor composition into the liquid hydrocarbon media, wherein the corrosion inhibitor composition includes

A-HN—(CH₂)₂—[—NH(CH₂)₂—]ₓ—NH-A I

or structure II:

A-HN—(CH₂)₃—[—O—(CH₂)₂—]ₙ—O—(CH₂)₃—NH-A II

wherein A has structure III:

R—CH═CH—C(CO₂H)—CH₂C(O)— III

and R is a (C₈-C₂₂) alkyl group or a (C₈-C₂₂) alkenyl group, x is an integer from 0 to 6 and n is an integer from 1 to 6.

The corrosion inhibitor forms a film on metal surfaces and protects the metal from corrosion caused by mineral or organic acids that may be contained in the liquid hydrocarbon media. The metallic surfaces may be any type of metal subject to acid corrosion. In one embodiment, the metal to be protected may be ferrous metals, such as steel or carbon steel, or non-ferrous metals, such as copper, bronze, brass, aluminum or titanium. In one embodiment, the metallic surfaces in contact with the liquid hydrocarbon media are found in hydrocarbon processing equipment, such as distillation units.

The liquid hydrocarbon media may be any type of liquid hydrocarbon media that may undergo processing. In one embodiment, the liquid hydrocarbon media includes, but is not limited to, crude oil, natural gas, condensate, heavy oil, processed residual oil, bituminous, coker oils, coker gas oils, fluid catalytic cracker feeds, gas oil, naphtha, FCC slurry, diesel fuel, fuel oil, jet fuel, gasoline, kerosene, crude styrene distillation tower feed, crude ethyl benzene column feed, pyrolysis gasoline, chlorinated hydrocarbons feed or vacuum residua.

In one embodiment, the corrosion inhibitor composition includes polyamic acid having structure I:

A-HN—(CH₂)₂—[—NH(CH₂)₂—]ₓ—NH-A I

or structure II:

A-HN—(CH₂)₃—[—O—(CH₂)₂—]ₙ—O—(CH₂)₃—NH-A II

wherein A has structure III:

R—CH═CH—C(CO₂H)—CH₂C(O)— III

and R is a (C₈-C₂₂) alkyl group or a (C₈-C₂₂) alkenyl group, x is an integer from 0 to 6 and n is an integer from 1 to 6.

In one embodiment, R is an alkyl group having from 8 to 22 carbon atoms. In another embodiment, R is a (C₁₀-C₂₀) alkyl group. In another embodiment, R is a (C₁₂-C₂₀) alkyl group. In another embodiment, R is a (C₁₄-C₂₀) alkyl group. In another embodiment, R is a (C₈-C₁₈) alkyl group. In another embodiment, R is a (C₁₂-C₁₈) alkyl group. Examples of suitable alkyl groups include, but are not limited to, decyl, dodecyl, tetradecyl, hexadecyl and octadecyl. The alkyl radical can be straight-chain or branched-chain. In one embodiment, the R group may be substituted.

In another embodiment, R is an alkenyl group having from 8 to 22 carbon atoms. In another embodiment, R is a (C₁₀-C₂₀) alkyl group. In another embodiment, R is a (C₁₂-C₂₀) alkenyl group. In another embodiment, R is a (C₁₄-C₂₀) alkenyl group. In another embodiment, R is a (C₈-C₁₈) alkenyl group. In another embodiment, R is a (C₁₂-C₁₈) alkenyl group. Examples of suitable alkenyl groups include, but are not limited to, decenyl, dodecenyl, tetradecenyl, hexadecenyl and octadecenyl. The alkenyl radical can be straight-chain or branched-chain. In one embodiment, the R group may be substituted.

In one embodiment, the aromatic amine group may be 1,4-aminophenyl.

In one embodiment, x is an integer from 0 to 6. In another embodiment, x is an integer from 1 to 2.

In one embodiment, n is an integer from 1 to 6. In another embodiment, n is an integer from 1 to 2.

In another embodiment, a corrosion inhibitor composition includes polyamic acid having structure VI:

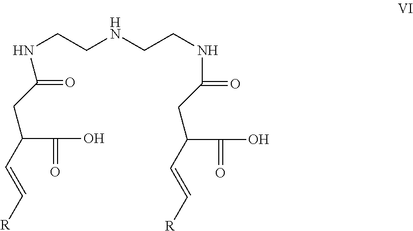

wherein R is described above and may be a (C₈-C₂₂) alkyl or (C₈-C₂₂) alkenyl group.

The corrosion inhibitor composition is dispersed within the liquid hydrocarbon media in any amount for forming a protective film on metallic surfaces in contact with the liquid hydrocarbon media. The corrosion inhibitor may be dispersed in the liquid hydrocarbon media in any manner suitable to mix or blend the corrosion inhibitor within the liquid hydrocarbon media. In another embodiment, the corrosion inhibitor composition may be dispersed within the liquid hydrocarbon media as the hydrocarbon media is transported through the processing equipment, such as a pipe or tube. In another embodiment, the corrosion inhibitor composition may be delivered in metered amounts into the liquid hydrocarbon media. In another embodiment, a feeding system may be used to add the corrosion inhibitor composition to the liquid hydrocarbon media. The feeding system may include a pump and a storage container. In another embodiment, the corrosion inhibitor composition may be injected into the liquid hydrocarbon media by a conventional in-line injection system and may be injected at any point in-line suitable to allow the corrosion inhibitor composition to mix with the liquid hydrocarbon media. The corrosion inhibitor composition may be added to the liquid hydrocarbon media in a continuous manner or can be added in one or more batch modes and repeated additions may be made.

In one embodiment, the corrosion inhibitor composition may be added to the liquid hydrocarbon media in a dosage amount of at least about 1 ppm by volume, based on the volume of the liquid hydrocarbon media. In another embodiment, the corrosion inhibitor composition may be added in an amount of at least about 5 ppm by volume, based on the volume of the liquid hydrocarbon media. In another embodiment, the corrosion inhibitor composition is present in an amount of from about 1 ppm by volume to about 200 ppm by volume, based on the volume of the liquid hydrocarbon media. In another embodiment, the corrosion inhibitor composition is added in an amount of from about 5 ppm by volume to about 100 ppm by volume, based on the volume of the liquid hydrocarbon media. In another embodiment, the corrosion inhibitor is added in an amount of from about 10 ppm by volume to about 50 ppm by volume, based on the volume of the liquid hydrocarbon media. In another embodiment, the corrosion inhibitor is present from about 10 ppm by volume to about 40 ppm by volume, based on the volume of the liquid hydrocarbon media.

In another embodiment, a method for making a corrosion inhibitor composition includes reacting an alkenyl succinic anhydride and a polyamine in a molar ratio of the anhydride to the polyamine of from about 1:1 to about 5:1 at a temperature from about 50° C. to about 95° C., wherein the polyamine has formula IV:

H₂N—(CH₂)₂—[—NH(CH₂)₂—]—NH₂ IV

or formula V:

H₂N—(CH₂)₃—[—O—(CH₂)₂)ₙ—O—(CH₂)₃—NH₂ V

wherein x is an integer from 0 to 6 and n is an integer from 1 to 6.

In one embodiment, the alkenyl succinic anhydride has structure VII:

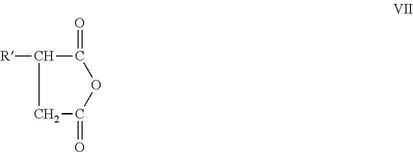

wherein R′ is a (C₈-C₂₂) alkenyl group.

Examples of the alkenyl succinic anhydride include, but are not limited to, octenyl succinic anhydride, 2-methylheptenyl succinic anhydride, nonenyl succinic anhydride, decenyl succinic anhydride, 5-methyl-2-isopropylhexenyl succinic anhydride, 1,2-dibromo-2-ethyloctenyl succinic anhydride, undecenyl succinic anhydride, 1,2-dicholoro-undecenyl succinic anhydride, 3-ethyl-2-t-butylpentenyl succinic anhydride, dodecenyl succinic anhydride, 2-propylnonenyl succinic anhydride, tridecenyl succinic anhydride, tetradecenyl succinic anhydride, hexadecenyl succinic anhydride, sulfurized octadecenyl succinic anhydride, octadecenyl succinic anhydride, 1,2-dibromo-2-methylpentadecenyl succinic anhydride, 8-propylpentadecenyl succinic anhydride; eicosenyl succinic anhydride, 2-octyldodecenyl succinic anhydride, and tetrapropenyl succinic anhydride.

Methods for preparing alkenyl succinic anhydrides are known in the art. In one example, a maleic anhydride is reacted with an olefin compound in equimolar proportions to form an alkenyl succinic anhydride.

In one embodiment, the polyamine may have formula IV:

H₂N—(CH₂)₂—[—NH(CH₂)₂—]—NH₂ IV

or formula V:

H₂N—(CH₂)₃—[—O—(CH₂)₂)ₙ—O—(CH₂)₃—NH₂ V

wherein x is an integer from 0 to 6 and n is an integer from 1 to 6. The integers x and n are described above. The polyamines may be aliphatic or cycloaliphatic.

Examples of polyamines include, but are not limited to, diethylenetriamine (DETA), triethylenetetraamine, ethylene diamine (EDA), hexamethylene diamine (HMDA), triethylene tetraamine (TETA), propylene diamine, diethylene triamine, trimethylene diamine, tripropylene tetramine, tetraethylene pentamine, hexaethylene heptamine, pentaethylenehexamine (PEHA), diethyleneaminopropylamine, m-phenylene-diamine, p-phenylenediamine, methylenedianiline, triethylene tetraamine, tetraethylene pentamine, melamine, piperazine, isopropylenediamine, butylenediamine, pentylenediamine, hexylenediamine, dipropylenetriamine, diisopropylenetriamine, dibutylenetriamine, di-sec-butylenetriamine, triethylenetetraamine, tripropylenetetraamine, triisobutylenetetraamine, tetraethylenepentamine, pentaethylenehexamine or dimethylaminopropylamine.

The molar ratio of the alkenyl succinic anhydride to the polyamine, is from about 1:1 to about 5:1. In another embodiment, the molar ratio is from about 1:1 to about 3:1. In another embodiment, the mole ratio is about 2:1. In another embodiment, the molar ratio is from about 3:1 to about 5:1.

The reactants are reacted at a temperature in a range of from about 50° C. to about 95° C. In another embodiment, the temperature is in a range of from about 50° C. to about 80° C. In another embodiment, the temperature is in a range of from about 60° C. to about 80° C.

In order that those skilled in the art will be better able to practice the present disclosure, the following examples are given by way of illustration and not by way of limitation.

### EXAMPLES

### Example 1

Synthesis of polyamic acid from DDSA (dodecenylsuccinic anhydride) and DETA (diethylene triamine). 15 ml of a heavy aromatic naphtha and 13.0 g (0.0488 moles) of DDSA were charged to a 100 ml three-neck round-bottom flask fitted with a condenser and a mechanical stirrer and the ingredients were stirred at room temperature under nitrogen atmosphere. To the stirred solution, 1 g of DETA (0.0097 moles) was added drop-wise at 40° C. This is an exothermic reaction and the reaction temperature was maintained at 60° C. during addition. After the complete addition of DETA, the reaction mixture was stirred for 3 hours at 80° C. The reaction mixture was cooled and the solid content was noted. The product was characterized by ¹³C NMR, FTIR, HPLC, LC-MS, water content and TAN number and identified as polyamic acid. The presence of a carbonyl peak due to —C═O—NH at 1604 cm⁻¹, 1554 cm⁻¹ and 1495 cm⁻¹ confirms the formation of amic acid in addition to presence of —COOH peak at 1707 cm⁻¹.

### Example 2

Synthesis of polyamic acid from DDSA and DETA. 15 ml of a heavy aromatic naphtha and 10.0 g (0.037 moles) of DDSA were charged to a 100 ml three-neck round-bottom flask fitted with a condenser and a mechanical stirrer and the ingredients were stirred at room temperature under nitrogen atmosphere. To the stirred solution, 1.93 g of DETA (0.0187 moles) was added drop-wise at 40° C. This is an exothermic reaction and the reaction temperature was maintained at 60° C. during addition. After the complete addition of DETA, the reaction mixture was stirred for 3 hours at 80° C. The reaction mixture was cooled and the solid content was noted. The product was characterized by ¹³C NMR, FTIR, HPLC, LC-MS, water content and identified as polyamic acid. The presence of a carbonyl peak due to —C═O—NH at 1604 cm⁻¹, 1554 cm⁻¹ and 1495 cm⁻¹ confirms the formation of amic acid in addition to presence of —COOH peak at 1707 cm⁻¹. The FIGURE shows the ¹³C NMR spectra for Example 2.

TAN is a measure of the diacid in the reaction product. The product was tested by Karl Fischer titration and shown to have an observed TAN of 166 mgKOH/g of sample. This TAN value correlates to a product having >97% amic acid and <3% diacid from the DDSA. Pure amic acid (100%) has an observed TAN of 172 mgKOH/g of sample.

### Example 3

A Rotating Cage Autoclave Corrosion Test was performed to measure the corrosion inhibiting properties of various samples. The test was performed in accordance with ASTM G170.

Test Fluid: 450 ml synthetic naphtha (10% cyclohexane, 10% toluene, 20% kerosene, 20% octane, 20% iso-octane and 20% heptane) was added to 50 ml brine containing 1100 ppm hydrochloric acid; 130 ppm sulfurous acid; 730 ppm sulfuric acid; 120 ppm acetic acid; 150 ppm propionic acid; 125 ppm butyric acid; 200 ppm pentanoic acid; 60 ppm hexanoic acid; 135 ppm ammonia and 50 ppm ammonium sulfide.

Experimental Conditions:

Test Temperature: 110° C.

Rotating speed: 1500 rpm

Atmosphere: Nitrogen

Corrosion specimen: Carbon steel (C 1010)

Sample 1 was prepared following Example 2. Sample 2 was prepared following the preparation in Example 1 and using a 4:1 molar ratio of anhydride to the polyamine. Sample 3 was prepared following Example 1. Sample 4 was prepared following Example 1 and substituting hexadecenyl succinic anhydride (commercial grade ASA-100) for the DDSA. Sample 5 was prepared following Example 1 and substituting an amino ether having the structure VIII:

NH₂—(CH₂)₃—O—CH₂—CH₂—O—(CH₂)₃—NH₂ VIII

for DETA and using a 4:1 molar ratio of anhydride to the polyamine. Sample 6 was prepared following Example 1, substituting an amino ether having structure VIII for DETA and using a 1.87:1 molar ratio of anhydride to the polyamine. Sample 7 was prepared following Example 1, substituting an amino ether having structure VIII for DETA and using a 4:1 molar ratio of anhydride to the polyamine. Sample 8 was prepared following Example 2, substituting an amino ether having structure VIII for DETA.

The samples were dispersed separately in the test fluid and the corrosion rate is shown in Table 1. A blank sample was run and the corrosion rate was 1012 mpy. The corrosion inhibition efficacy tests were measured at pH of 2 and pH of 5.2.

TABLE 1

| | | | | | | Corrosion |
|---|---|---|---|---|---|---|
| | | | | | | Inhibition |
| | | | | | | efficiency |
| | | | | Corrosion Rate | | for |
| | | | Molar | (mpy) | | 10 ppmA |
| Sample | Anhydride | Polyamine | Ratio | 5 ppmA | 10 ppmA | (%) |
| 1 | DDSA | DETA | 2:1 | N/A | 39 | 96 |
| 2 | DDSA | DETA | 4:1 | 102 | N/A | N/A |
| 3 | DDSA | DETA | 5:1 | 17 | 30 | 97 |
| 4 | DDSA | VII¹ | 4:1 | 638 | N/A | N/A |
| 5 | DDSA | VII | 1.9:1 | N/A | 15 | 98 |
| 6 | ASA² | VII | 2:1 | N/A | 29 | 97 |
| 7 | ASA | VII | 4:1 | 776 | 16 | 98 |
| 8 | ASA | DETA | 5:1 | N/A | 18 | 98 |

¹VII is structure VII shown above.
²ASA is hexadecenyl succinic anhydride (commercial grade ASA-100)

All samples measured at 10 ppmA provided excellent corrosion inhibition efficiency rates at low dosage amounts.

### Example 4

200 ppm sulfur was added to the Test Fluid in Example 3 and the corrosion rate of Sample 1 was tested by the Rotating Cage Autoclave Corrosion test shown in Example 3. The results are shown in Table 2. The corrosion rate was measured at a pH of 5.2.

TABLE 2

| | | | Molar | Corroion rate at 20 |
|---|---|---|---|---|
| Sample | Anhydride | Polyamine | Ratio | ppmA (mpy) |
| 1 | DDSA | DETA | 2:1 | 2 |
| Blank | N/A | N/A | N/A | 683 |

Sample 1 shows excellent corrosion inhibition properties.

### Example 5

8 ppm of dissolved oxygen was added to the Test Fluid in Example 3 and the corrosion rate of Sample 1 was tested by the Rotating Cage Autoclave Corrosion test shown in Example 3. The results are shown in Table 3. The corrosion rate was measured at a pH of 5.2.

TABLE 3

| | | | Molar | Corroion rate at 20 |
|---|---|---|---|---|
| Sample | Anhydride | Polyamine | Ratio | ppmA (mpy) |
| 1 | DDSA | DETA | 2:1 | 2 |
| Blank | N/A | N/A | N/A | 245 |

Sample 1 shows excellent corrosion inhibition properties.

### Example 6

The Rotating Cage Autoclave Corrosion test shown in Example 3 was repeated for Sample 1 at a dosage amount of 40 ppmA. The corrosion rate for a blank sample was measured at 1515 mpy at pH of 2. The corrosion rate for Sample 1 was measured as 13, which is a 98.5% corrosion inhibition efficiency.

1. A method for inhibiting corrosion on metallic surfaces in contact with a liquid hydrocarbon media, the method comprising dispersing a corrosion inhibitor composition into the liquid hydrocarbon media, wherein the corrosion inhibitor composition comprises polyamic acid having structure I:
A-HN—(CH₂)₂—[—NH(CH₂)₂—]ₓ—NH-A I
or structure II:
A-HN—(CH₂)₃—[—O—(CH₂)₂—]ₙ—O—(CH₂)₃—NH-A II
wherein A has structure III:
R—CH═CH—CH(CO₂H)—CH₂C(O)— III
and R is a (C₈-C₂₂) alkyl group or a (C₈-C₂₂) alkenyl group, x is an integer from 0 to 6 and n is an integer from 1 to 6.

2. The method of claim 1 wherein the liquid hydrocarbon media comprises crude oil, natural gas, condensate, heavy oil, processed residual oil, bituminous, coker oils, coker gas oils, fluid catalytic cracker feeds, gas oil, naphtha, FCC slurry, diesel fuel, fuel oil, jet fuel, gasoline, kerosene, crude styrene distillation tower feed, crude ethyl benzene column feed, pyrolysis gasoline, chlorinated hydrocarbons feed or vacuum residua.

3. The method of claim 1, wherein the corrosion inhibitor composition comprises polyamic acid having structure VI: 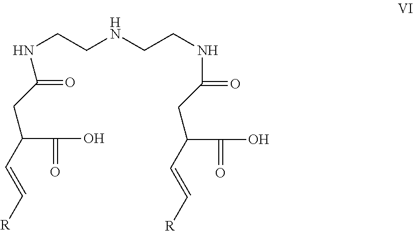
wherein R is a (C₈-C₂₂) alkyl group or a (C₈-C₂₂) alkenyl group.

4. The method of claim 1, wherein the corrosion inhibitor composition is added to the liquid hydrocarbon media in a dosage amount of at least about 1 ppm by volume to about 200 ppm by volume, based on the volume of the liquid hydrocarbon media.

5-13. (canceled)
